(19) United States
(12) Patent Application Publication
(10) Pub. No.: US 2003/0186220 A1
(43) Pub. Date: Oct. 2, 2003

(54) Detecting microorganisms using whole genomic DNA or RNA microarray

(76) Inventors: Jizhong Zhou, Oak Ridge, TN (US); Dorothea Kathleen Thompson, Knoxville, TN (US); Liyou Wu, Oak Ridge, TN (US)

Correspondence Address: Zhibin Ren, Quarles & Brady LLP, 1 South Pinckney Street, P O Box 2113, Madison, WI, 53701-2113, US

(21) Appl. No.: 10/112,636

(22) Filed: Mar. 28, 2002

(51) Int. Cl. C12Q 1/70; C12Q 1/68

(52) U.S. Cl. 435/5; 435/6

(57) ABSTRACT

The present invention is a method for determining the presence or absence of a microorganism in a sample. The method involves providing a nucleic acid microarray having one or more probes that represent at least a substantial portion of the whole genomic DNA or RNA of the microorganism, hybridizing a labeled DNA or RNA preparation derived from the sample to the microarray, washing the microarray, and observing the presence or absence of a hybridization signal at the position where the one or more probes are located to determine the presence or absence of the microorganism in the sample.

### STATEMENT REGARDING FEDERALLY SPONSORED RESEARCH OR DEVELOPMENT

This invention was made with United States government support awarded by the following agency: DOE, Grant No. KP1301010. The United States has certain rights in this invention.

### CROSS-REFERENCE TO RELATED APPLICATIONS

Not applicable.

### BACKGROUND OF THE INVENTION

Many microorganisms affect human health, environmental quality, and agricultural and industrial processes. Methods for detecting microorganisms of interest that are rapid, field-applicable, sensitive, quantitative and high throughput are desirable for addressing microbial problems associated with human health (e.g., pathogen detection in humans, microbial ecology of infectious diseases), animal (e.g., intestinal and rumen) productivity and health, plant (e.g., rhizosphere) growth and health, water and food safety (pathogen detection in water and food sources), forestry, oceanography, fisheries, biodiversity discovery and management (e.g., pharmaceutical discovery), bioprocessing of industrial products, waste-water treatment, and bioremediation of environmental contaminants. Such methods will also help in understanding the structure and composition of microbial communities and their responses to environmental perturbations such as toxic contamination, climate change, and agricultural and industrial practices. This understanding is critical for the maintenance and restoration of desirable ecosystem functions.

Current available methods for detecting the presence of microorganisms are either culture-based or culture-independent. Since more than 99% of microorganisms are hard to culture (Amann et al., 1995), culture-independent methods can be advantageous in many situations. Examples of culture-independent methods include 16S rRNA gene-based cloning methods, denaturing gradient gel electrophoresis or DGGE, T-RFLP, and quantitative PCR (Amann et al., 1995).

Reverse Sample Genome Probing (RSGP) is another culture-independent method. It is based on genomic DNA hybridization and permits simultaneous detection and quantitation of selected bacteria from environmental samples (Voordouw et al., 1991). RSGP has been employed in oil fields (Voordouw et al., 1991; 1992; 1993), terrestrial soils (Shen et al., 1998), and intertidal salt marsh sediments (Bagwell and Lovell, 2000) to monitor changes in the representation of sulfate reducer and nitrogen fixer populations, respectively, in response to environmental variability. However, RSGP is extremely time consuming, labor intensive, and only permits examination of relatively small subsets of microorganisms. Improvements of existing culture-independent methods or the development of new methods are desirable.

Numerous recent studies have demonstrated the utility of microarrays for analyzing gene expression and regulation on a genomic scale (e.g., Brocklehurst and Morby, 2000; DeRisi et al., 1997; DeRisi et al., 1996; de Saizieu et al., 1998; Drmanac et al., 1996; Ferea et al., 1999; Futcher, 2000; Gasch et al., 2000; Gross et al., 2000; Heller et al., 1997; Khodursky et al., 2000; Lashkari et al., 1997; Lockhart et al., 1996; Lyons et al., 2000; Milosavljevic et al., 1996; Peterson et al., 2000; Richmond et al., 1999; Schena et al., 1995; Schena et al., 1996; Selinger et al., 2000; Sudarsanam et al., 2000; Wei et al., 2001; White et al., 1999; Wodicka et al., 1997; Ye et al., 2000; Zhang et al., 1997) and for detection of genetic polymorphisms (Chee et al., 1996; Hacia, 1999; Wang et al., 1998) in both eukaryotes and prokaryotes. Compared to conventional membrane-based hybridization methods, glass slide-based microarrays offer the advantages of high-throughput sample analysis, rapid detection, low background levels, and high sensitivity (Shalon et al., 1996).

Current applications of DNA microarray technology require prior knowledge of nucleotide sequence information for microarray fabrication. At the same time, studies involving microarrays have so far been limited to using relatively pure samples such as pure cultures and it is not clear whether DNA microarray technology can be successfully used for nucleic acid preparations derived from environmental samples. In contrast to studies using pure cultures, microarray-based analysis of environmental nucleic acids presents a number of problems. In environmental studies, the target and probe sequences can be very diverse, and it is not clear whether the performance of microarrays with diverse environmental samples is similar to that with pure culture samples and how sequence divergence affects microarray hybridization. Also, environmental samples generally contain other substances, such as humic materials, organic contaminants and metals that may inhibit DNA hybridization on microchips. In addition, unlike pure cultures, the biomass in environmental samples is generally low. It is not clear whether microarray hybridization is sensitive enough for detecting microorganisms in environmental samples. Finally, it is uncertain whether microarray-based detection can be quantitative.

### BRIEF SUMMARY OF THE INVENTION

The present invention relates to determining the presence or absence of a microorganism of interest using microarray technology. In one embodiment, the present invention is a method for determining the presence or absence of a microorganism in a sample. The method involves providing a nucleic acid microarray having one or more probes that represent at least a substantial portion of the whole genomic DNA or RNA of the microorganism, hybridizing a labeled DNA or RNA preparation derived from the sample to the microarray, washing the microarray, and observing the presence or absence of hybridization between the preparation and the probe to determine the presence or absence of the microorganism in the sample. In another embodiment, the present invention is the nucleic acid microarray as described above. In a third embodiment, the present invention is a method of building the nucleic acid microarray of the present invention by isolating the genomic DNA or RNA of the microorganism and spotting it onto a microarray substrate.

It is a feature of the present invention that the probes on the nucleic acid microarray used for detecting microorganisms represent the whole or substantially the whole genomic DNA or RNA of the microorganisms.

It is an advantage of the present invention that no sequence information of a microorganism is necessary for detecting that microorganism.

It is another advantage of the present invention that microorganisms in complex environmental samples can be detected.

It is another advantage of the invention that the method for detecting microorganisms is high throughput.

It is another advantage of the present invention that the method for detecting microorganisms is sensitive and can be quantitative as well.

Other objects, advantages, and features of the present invention will become apparent from the following specification when taken in conjunction with the accompany drawings.

### BRIEF DESCRIPTION OF THE SEVERAL VIEWS OF THE DRAWINGS

FIG. 1 is a graph illustrating the effect of different formamide concentrations in the hybridization solution on hybridization signal intensity.

FIG. 2 shows the relationship between target DNA concentration and hybridization signal intensity.

FIG. 3 shows the result of using the DNA microarray method of the present invention to detect microorganisms in diverse environmental samples.

### DETAILED DESCRIPTION OF THE INVENTION

The present invention teaches that one can use a nucleic acid microarray containing one or more probes that represent the whole or a substantial portion of the whole genomic DNA or RNA of a microorganism to detect the presence or absence of the microorganism in a sample by preparing a DNA or RNA preparation from the sample and hybridizing the DNA or RNA preparation to the microarray. A positive hybridization indicates the presence of a microorganism. The method has been proven to work for complex environmental samples. Since a nucleic acid microarray can accommodate many probes, the method allows detecting the presence or absence of many different microorganisms simultaneously. The method is specific in that species within a genus or strains within a species can be distinguished. The method is also sensitive in that as low as 0.2 ng genomic DNA of a microorganism is sufficient for detecting the microorganism. In addition, the method can be made quantitative. Since the method uses the whole or a substantial portion of the whole genomic DNA or RNA as probes, no sequence information is necessary for detecting the presence of a microorganism.

In addition to the method described above, the microarray itself and methods for building the microarray by isolating and spotting the whole or a substantial portion of the whole genomic DNA or RNA onto a microarray substrate are also within the scope of the present invention.

In the present invention, the probes on a nucleic acid microarray for detecting the presence of a microorganism represent the whole or a substantial portion of the whole genomic DNA or RNA of the microorganism. By a substantial portion of the whole genomic DNA or RNA of a microorganism, we mean at least 90% of the whole genomic DNA or RNA. Preferably, the probes are at least 95%, and most preferably at least 99% of the whole genomic DNA or RNA of the microorganism.

The whole or a substantial portion of the whole genomic DNA or RNA of a microorganism can be represented by one or more probes. For example, when a bacterium has three chromosomes, the whole or a substantial portion of the whole genomic DNA of the bacterium is represented by at least three probes. If the whole genomic DNA is sheared by ultrasound or other physical means during the isolation process, the whole or a substantial portion of the whole genomic DNA of the bacterium will be represented by many more probes. Regardless how many probes are used to represent the whole or a substantial portion of the whole genomic DNA or RNA of a microorganism, these probes are spotted within an area of a microarray substrate that is considered to be one single position on the microarray and the total hybridization signal of the position is used to determine the presence or absence of a microorganism in a sample. Thus, a position of a microarray is herein defined as an area on the microarray the hybridization signals from which are detected as a whole. Obtaining genomic DNA or RNA from microorganisms and spotting the obtained DNA or RNA onto a nucleic acid microarray substrate are well within the capability of one of ordinary skill in the art. In the examples below, a method for isolating genomic DNA and building a DNA microarray with the isolated genomic DNA is described. Other methods known in the art can also be used.

The microarray probes for detecting microorganisms in the present invention may contain short nucleotide sequences (for example less than 100 nucleotides) that are not native to the microorganisms so long as these sequences do not interfere with the detection of the microorganisms. These sequences may but do not have to serve certain functions such as facilitating the attachment of the probes to a microarray substrate.

In the present invention, the DNA or RNA preparation derived from a sample that is used to hybridize a microarray can be a whole genomic DNA or RNA preparation, or a cDNA or mRNA preparation. As long as the DNAs or RNAs in the preparation represent the whole or a substantial portion of the whole genomic DNA or RNA, or cDNA or mRNA, the preparation can be used to hybridize the microarray for detecting the presence of a microorganism of interest in the sample. For example, the DNA or RNA preparation can be digested with a restriction enzyme and the resultant preparation containing smaller pieces of DNA or RNA is still useful for detecting the presence of a microorganism of interest in the sample.

In the present invention, the DNA or RNA molecules in the DNA or RNA preparation derived from a sample are labeled to facilitate hybridization detection. Methods and materials that can be used to label DNA or RNA molecules are known in the art. In the examples below, a method of labeling the whole genomic DNA with Cy3 or Cy5 is described. However, other known labeling materials and methods may also be used. For example, other Cy dyes such as Cy3.5 and Cy5.5, Alexa fluorescent dyes, and radioactive isotopes such as ³³P can also be used to label the whole genomic DNA to facilitate the detection of hybridization. Furthermore, a two-color fluorescent labeling strategy may be used in the present invention. The strategy is described in Ramsay 1998 and Shalon et al. 1996, both of which are incorporated by reference in their entireties. Such multiple-color hybridization detection strategy minimizes variations resulting from inconsistent experimental conditions and allows direct and quantitative comparison of target abundance among different samples (Ramsay, 1998 and Shalon et al., 1996).

Examples of hybridization and washing conditions that can be used in the present invention are described in the examples below. It has been shown that including a denaturant in the hybridization buffer increased hybridization specificity. Higher denaturant concentration led to higher hybridization specificity. Examples of hybridization denaturants that can be used include but are not limited to formamide and dimethyl sulfoxide. However, using a denaturant is not mandatory and hybridization stringency can be adjusted through other means. When formamide is used, its preferred concentration in a hybridization buffer is from about 5% to about 70%, from about 30% to about 70%, or from about 50% to about 70%, although concentrations outside the above ranges can also be used. The term “about” is used in the specification and claims to cover concentrations and temperatures that vary a little from the recited concentration and temperature but retain the essential function of the recited concentration and temperature. The effect of hybridization temperature and salt concentration in the washing buffer on hybridization specificity were also tested in the examples below. Hybridization temperatures tested ranged from 45° C. to 75° C. and salt concentrations tested ranged from 0×SSC to 1×SSC. Although other temperatures and salt concentrations can be used, the preferred hybridization temperature for the particular applications in the examples below is from about 45° C. to about 75° C. or from about 55° C. to about 75° C., and the preferred salt concentration in the washing buffer is from 0×SSC to about 0.1×SSC, from 0×SSC to about 0.05×SSC or from 0×SSC to about 0.01×SSC.

The examples below show that suitable hybridization and washing conditions depend on specific applications. The higher the degree of similarity between two microorganisms that need to be differentiated, the higher the stringency of hybridization and washing conditions should be. For example, when an application calls for differentiating microorganisms on the genus level, hybridization and washing conditions of relatively low stringency may do the job. When an application calls for differentiating microorganisms on the species level wherein the microorganisms share a higher degree of similarity, hybridization and washing conditions of higher stringency are required. When an application calls for differentiating microorganisms on the strain level wherein the microorganisms share an even higher degree of similarity, hybridization and washing conditions of even higher stringency are required. Now that the present invention has shown that microarray-based whole genomic DNA to whole genomic DNA hybridization works, one of ordinary skill in the art can readily determine suitable hybridization and washing conditions for a particular application. A higher denaturant concentration in the hybridization buffer, a higher hybridization temperature, a lower SSC concentration in the washing buffer, a higher washing temperature, or a combination of any of the above will provide a higher hybridization and washing stringency.

It should be noted that the hybridization and washing conditions provided in the examples below are only examples and other hybridization and washing conditions may also work. For example, a different hybridization or washing buffer may be able to replace the buffers described in the examples. Temperature and concentration ranges outside those tested in the examples may also be acceptable for a particular application. One of ordinary skill in the art knows about these other hybridization and washing conditions and how to modify these conditions in order to achieve a desired degree of specificity. For example, nucleic acid duplex or hybrid stability is expressed as the melting temperature or Tm, which is the temperature at which a probe dissociates from a target DNA. This melting temperature is used to define the required stringency conditions. If sequences are to be identified that are related and substantially identical to the probe, rather than identical, then it is useful to first establish the lowest temperature at which only homologous hybridization occurs with a particular concentration of salt (e.g., SSC or SSPE). Then, assuming that 1% mismatching results in a 1° C. decrease in the Tm, the temperature of the final wash in the hybridization reaction is reduced accordingly. In practice, the change in Tm can be between 0.5° C. and 1.5° C. per 1% mismatch. Additional guidance regarding such conditions is readily available in the art, for example, by Sambrook et al., 1989, Molecular Cloning, A Laboratory Manual, Cold Spring Harbor Press, N.Y.; and Ausubel et al. (eds.), 1995, Current Protocols in Molecular Biology, (John Wiley & Sons, N.Y.) At Unit 2.10.

After hybridization and washing, the presence or absence of hybridization signals on the microarray is determined. The nature of the hybridization signal and the method of detecting it depend on the labeling material used for the DNA or RNA derived from a sample. For example, when the DNA is labeled with Cy3 or Cy5, fluorescence at 570 nm or 670 nm, respectively, is the hybridization signal and can be detected accordingly. The presence of a hybridization signal at a particular position on the microarray indicates that the microorganism represented by the probes at the position existed in the sample.

The examples below show that the log value of fluorescence intensity and the log value of the target DNA concentration displayed a linear relationship over a wide range of target DNA concentrations: from 0.067 ng/μl to 667 ng/μl. Thus, the method of the present invention can be used to compare the relative abundance of two or more microorganisms in a sample. In addition, with proper standard curve set up, the method of the present invention can also be used to quantitate the amount of a microorganism in a sample.

The present invention can be used to determine the presence or absence of a microorganism in any sample. Examples include but are not limited to samples obtained from the environment, samples obtained from a human being or a non-human animal, and samples obtained from a water or food source. With a sample obtained from the environment, the existence of one or more microorganisms in the environment can be determined. Further, it is also possible to determine the composition and structure of a microbial community in the environment. With a sample obtained from the human or non-human animal, whether the animal has been infected by one or more pathogens can be determined. Along the same line, a water or food source sample allows the determination as to whether the source has been contaminated with certain pathogens. Any microorganism whose genomic DNA or RNA can be isolated and spotted onto a microarray substrate can be a detection target using the method of the present invention. Examples of such microorganisms include but are not limited to viruses, bacteria, yeasts, fungi and algae.

By way of example, but not limitation, examples of the present invention are described below.

### EXAMPLES

Materials and Methods

Bacterial strains and environmental samples. Type strains and purified environmental isolates used in this study are given in Table 1. Shewanella and Pseudomonas reference organisms and aromatic compound-degrading Azoarcus isolates were obtained from our culture collections at the Oak Ridge National Laboratory and Michigan State University. Genotypic and phenotypic studies describing the taxonomic classification of these bacteria have been described elsewhere (Rossello et al., 1991; Song et al., 1999; Venkateswaran et al., 1999). Environmental isolates were collected from Washington continental margin sediments (Braker et al., 2000) or deep ocean marine sediments.

To evaluate the application of community genome arrays, samples from soil, stream sediments, and marine sediments were used. Soil (NC/A1, WBE/A1, and WBW/A1) and stream sediment (NC/S1, WBE/S1, and WBW/S1) samples were collected from field research sites located on the Oak Ridge National Laboratory Reservation in eastern Tennessee. Marine sediment samples (w303/1-1.5, w305/2-3, w305/9-10, w306/3-4, w307/1-1.5, and w307/9-10) were provided by Dr. Allan Devol at The University of Washington. E. coli genomic DNA was extracted from E. coli strain S17-1/λpir (Kalogeraki and Winans, 1997). Yeast genomic DNA was prepared from Saccharomyces cerevisiae ATCC 18824.

Genomic DNA purification and quantitation. Reference genomes arrayed on glass slides were isolated from pure cultures using previously described protocols (Sambrook et al., 1989). All genomic DNA samples were treated with RNase A (Sigma, St. Louis, Mo.) and analyzed on agarose gels stained with ethidium bromide prior to microarray fabrication. Community DNA from soil, stream sediment, and marine sediment samples was extracted according to the method described by Zhou et al. (1996), which is incorporated by reference in its entirety. DNA concentration was determined in the presence of ethidium bromide by fluorometric measurement of the excitation at 360 nm and emission at 595 nm using a HTS700 BioAssay Reader (Perkin Elmer, Norwalk, Conn.).

Microarray construction and post-processing. Initially, arrays consisting of whole genomic DNA from Shewanella algae BrY, Shewanella sp. MR-4, Shewanella pealeana ANG-SQ1, Azoarcus tolulyticus Td-15, Escherichia coli, and Saccharomyces cerevisiae were constructed to determine the effect of DNA probe concentration on hybridization signal intensity. Genomic DNA probes were printed onto silane-modified glass slides at concentrations of 10, 50, 100, 200, 300, 400, 500, 600, and 700 ng/ml. Fluorescence intensities became saturating for the target genome at DNA concentrations of 200 ng/ml or greater. Genome probe concentrations of 200 ng/ml were therefore used for construction of the prototype community genome arrays.

Community genome arrays contained reference genomic DNA from the following microorganisms (see Table 1): (1) 7 Shewanella species, 4 isolates of Azoarcus evansii and 3 isolates of A. tolulyticus, 9 Pseudomonas stutzeri strains, P. balearica, E. coli, and Saccharomyces cerevisiae from pure cultures; and (2) 28 isolates from environmental samples, including 9 Shewanella species, 7 P. stutzeri species, 4 Marinobacter sp., 2 Bacillus methanolicus, 2 Azoarcus-like species, Staphylococcus saprophyticus, Halomonas variabilis, an unidentified α proteobacterium and marine bacterium. Five yeast genes encoding mating pheromone α-factors (mfα1, mfα2), mating-type α-factor pheromone receptor (ste3), actin (act1), and GTP-binding protein involved in the regulation of cAMP pathway (ras1) served as negative controls. To avoid confusion, the DNA deposited on the glass slides is referred to as the probe, whereas the fluorescently labeled DNA is designated as the target.

Genomic DNA samples were diluted to a final concentration of 200 ng/ml in 50% dimethyl sulfoxide (DMSO; Sigma, St. Louis, Mo.) and printed with a single pin (ChipMaker 3, TeleChem International, Sunnyvale, Calif.) at a spacing distance of 250 mm on silane-coated 25 mm×75 mm glass slides (Cel Associates, Inc., Houston, Tex.) using a PixSys 5500 robotic printer (Cartesian Technologies, Inc., Irvine, Calif.). All 59 probes were arranged as a matrix of 15 rows×4 columns. The exact location of each DNA element in the array matrix is listed in Table 1. Each glass slide contained 3 replicates of the community genome array.

Following printing, glass slides were post-processed as described previously (Wu et al., 2001, which is incorporated by reference in its entirety). To evaluate the quality of printing and the retention of arrayed DNA elements, a single slide from the same printed set of slides was stained for 30 min in a solution of PicoGreen (Molecular Probes, Eugene, Oreg.), diluted 1:200 in 1× TE buffer (10 mM Tris-HCl [pH 8.0] and 1 mM EDTA). The slide was then washed consecutively in 1× TE, 0.5× TE, and sterile dH₂O for 1 min each prior to being scanned using the ScanArray® 5000 Microarray Analysis System (GSI Lumonics, Watertown, Mass.).

Preparation of fluorescently labeled whole genomic DNA. To determine whether hybridization signal intensity could be improved by reducing the complexity of the labeled target, genomic DNA was fragmented using Sau3A restriction digestion and purified by ethanol precipitation. Poor hybridization results, however, were obtained using labeled Sau3A-digested genomic DNA. As a result, all later microarray experiments were performed using labeled whole genomic DNA.

Whole genomic DNA (2 μg) was denatured by boiling for 2 min and immediately chilled on ice for labeling. Each labeling reaction contained the following components in a total volume of 40 ml: denatured genomic DNA; 1× React 2 buffer (Gibco BRL, Gaithersburg, Md.); 1.5 μg of random hexamers (Gibco BRL) as primers; 50 mM dATP, dTTP, and dGTP; 20 mM dCTP; 10 mM of dCTP tagged with the fluorescent dye Cy3 (green pseudocoloror) or the fluorescent dye Cy5 (red pseudocolor) [NEN Life Science Products, Boston, Mass.]; and 10 U of the large Klenow fragment of DNA polymerase I (Gibco BRL). The reaction mixture was incubated at 37° C. for 2 h, heat-treated in a 100° C. heating block for 3 min, and chilled on ice. Labeled target DNA was purified immediately using the QIAquick PCR purification kit (Qiagen, Chatsworth, Calif.) according to the manufacturer's instructions, concentrated in a Savant SC110 Speedvac (Savant Instruments, Inc., Holbrook, N.Y.) at 40° C. for 1.5 h, and resuspended in 10 ml of dH₂O for hybridization, except for sensitivity experiments in which the labeled target DNA was resuspended in 3 ml of dH₂O.

Microarray hybridization. All microarray experiments were performed in triplicate (a total of 9 replicates per genomic DNA probe), unless otherwise noted. Hybridization solutions contained denatured fluorescently labeled genomic DNA, 3×SSC (1×SSC contained 150 mM NaCl and 15 mM trisodium citrate), 1 μg of unlabeled herring sperm DNA (Promega, Madison, Wis.), and 0.3% SDS in a total standard volume of 15 ml. Formamide was added to the hybridization solution for experiments testing the effect of a denaturant on hybridization specificity. A reduced hybridization solution volume of 3 ml or 5 ml was used, respectively, for testing detection sensitivity and analyzing environmental samples. In this case, the hybridization solution was deposited directly onto the immobilized DNA prior to placing a coverslip (6.25 mm×8 mm) over the array.

For detection sensitivity and quantitation experiments, hybridization was carried out under a coverslip in a waterproof CMT-slide chamber (Corning, Corning, N.Y.) submerged in a 65° C. water bath for 12-15 h. Prior to hybridization, fifteen microliters of 3×SSC was dispensed into the hydration wells on either side of the microarray slide in the slide chamber. For microarray experiments evaluating the effect of different formamide concentrations on hybridization specificity, hybridization was performed at 55° C. in the presence of 0, 10, 20, 30, 40, 50, 60, or 70% (vol/vol) formamide. For experiments determining the effect of temperature and denaturants on signal intensity, hybridization was carried out at 45, 55, 65, or 75° C. in the presence or absence of 50% (vol/vol) formamide. For microarray analysis of diverse environmental samples, hybridization was performed at 55° C. in the presence of 50% formamide. Following hybridization, coverslips were removed in washing buffer (1×SSC-0.2% SDS) and then washed sequentially for 5 min in 1×SSC-0.2% SDS and 0.1×SSC-0.2% SDS and for 30 sec in 0.1×SSC at ambient temperature prior to being air-dried in the dark. For experiments testing the effect of different salt concentrations in post-hybridization washing on microarray hybridization signals, slides were washed in the following solutions: (1) 1×SSC-0.2% SDS (5 min); (2) 0, 0.01×, 0.05×, 0.1×, or 0.5×SSC-0.2% SDS (10 min at each salt concentration); and (3) 0, 0.01×, 0.05×, 0.1×, or 0.5×SSC-0% SDS (30 sec).

Array scanning and quantitative analysis of hybridization signals. Glass slide microarrays were scanned at a resolution of 5 μm using the confocal laser microscope of the ScanArray® 5000 System. A separate scan using the appropriate excitation line (570 nm for Cy3 and 670 nm for Cy5) was performed depending on the fluorophore used. For sensitivity experiments and analysis of environmental samples, the laser power and photomultiplier tube (PMT) gain were both 100%. For specificity experiments, the laser power was 85% and the PMT gain was 75%.

The scanned image displays were saved as 16-bit TIFF files and analyzed by quantifying the pixel density (intensity) of each hybridization spot using the software of ImaGene® version 3.0 (Biodiscovery, Inc., Los Angeles, Calif.). A grid of individual circles defining the location of each DNA spot on the array was superimposed on the image to designate each fluorescent spot to be quantified. Mean signal intensity was determined for each spot. The local background signal was subtracted automatically from the hybridization signal of each separate spot. Fluorescence intensity values for the five yeast genes (negative controls) were averaged and then subtracted from the final quantitation values for each hybridization signal. Statistical analysis was performed using SigmaPlot 5.0 (Jandel Scientific, San Rafael, Calif.).

Results

Specificity of CGA hybridization. Microarrays consisting of genomic DNA isolated primarily from three major bacterial genera (Pseudomonas, Shewanella, and Azoarcus), including different selected species and strains of each (see Table 1), were constructed to examine hybridization specificity under varying experimental conditions and to determine threshold levels for phylogenetic differentiation. The effect of temperature, formamide, and salt concentration on hybridization specificity was assessed with A. evansii strain Td21 as the target template. Microarray hybridizations were performed in triplicate with a total of nine replicates per genome.

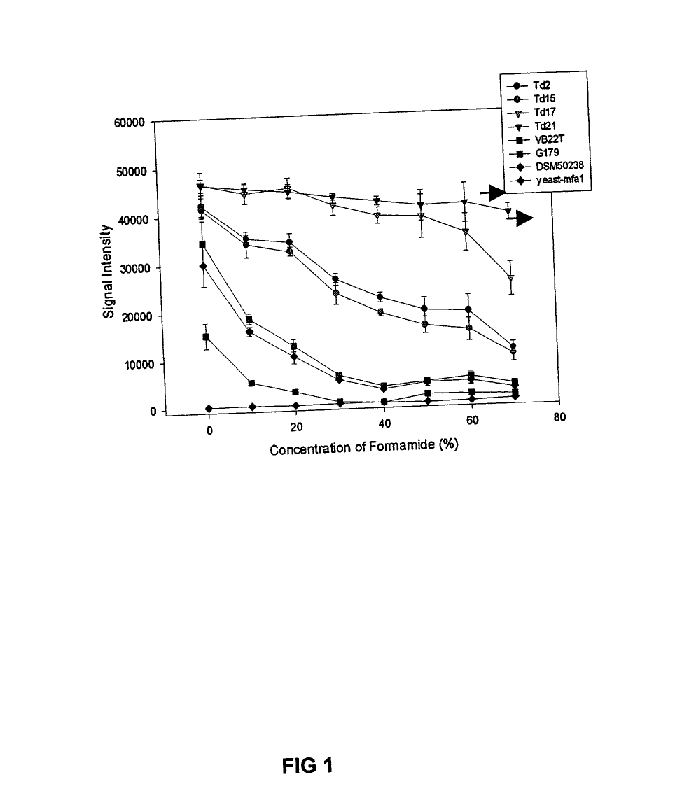

Increasing concentrations of formamide (ranging from 0 to 70%) in the hybridization buffer clearly had a substantial impact on hybridization specificity at 55° C. with Cy5-labeled Td21 genomic DNA (FIG. 1: data points represent mean values derived from 9 replicates for each arrayed genome; bars indicate the standard deviation of signal intensity). At low formamide concentrations (0 and 10%), extensive, non-specific cross-hybridization was observed between A. evansii Td21 and the majority of the P. stutzeri strains represented on the array, as well as VB22T, Marinobacter sp., and Staphylococcus saprophyticus. Hybridization specificity was greatly improved at formamide concentrations of 30 to 40%, with the labeled target genome (Td21) cross-reacting only with Azoarcus tolulyticus and other A. evansii strains. The percent DNA similarity of the majority of Td isolates, including BL-11, to the Td21 genome fell within the range of 27 to 33 (Table 2). Non-specific cross-hybridization was reduced to nearly background levels at formamide concentrations of 50 to 70%; cross-reaction with A. evansii Td17, which shares 89% genome similarity with Td21 based on hybridization methods (Table 2; Song et al., 1999), was detected under the hybridization conditions used.

The effect of temperature at a formamide concentration of 50% was examined to determine whether hybridization specificity could be improved. Microarray hybridizations were conducted at 55°, 65°, and 75° C. Compared to hybridization results at 55° C., the fluorescence intensities for all measurable signals decreased exponentially at 65° C. and again slightly at 75° C. CGA hybridization conditions of 50% (vol/vol) formamide at 55° C. were therefore selected for later experiments. Different salt (SSC) concentrations in post-hybridization washing were also evaluated. Decreasing the salt concentration in the wash buffer to less than 0.1× (i.e., 0.05×, 0.01×, or 0×) substantially reduced the degree of non-specific cross-hybridization without significantly affecting the target signal intensity; however, the use of 0.01×SSC in the wash buffer minimized the variability in signal intensity observed among replicates.

With the hybridization conditions of 50% formamide at 55° C., the specificity of genome:genome hybridizations on glass-based microarrays was investigated further using various fluorescently labeled target templates. The genomes of species within a genus (e.g., Pseudomonas and Shewanella) were clearly distinctive. Pseudomonas sp. G179 DNA, for example, did not cross-hybridize with the P. stutzeri genomes on the array, and Shewanella oneidensis MR-1 could be distinguished from other Shewanella species. Different strains of P. stutzeri were not clearly resolved (e.g., strains B2-2, E4-2, and ATCC 17587) under the conditions used. Complete specificity was observed for an unknown α-proteobacterium (C1-4) and Halomonas variabilis (B9-12), which shared no close phylogenetic relatives on the microarray.

Detection sensitivity of CGA hybridization. The detection sensitivity of hybridization with the community genome array was determined using genomic DNA from a pure culture of P. stutzeri isolate B2-2. The B2-2 genomic DNA was randomly labeled with Cy3 at concentrations that varied between 0.1 and 2000 ng. At a hybridization temperature of 65° C. in the absence of formamide, strong hybridization signals were observed with 5 ng of B2-2 genomic DNA for the target genome. With 0.2 ng of DNA, the target hybridization signal was substantially weaker but detectable. Hybridization signals using 0.1 ng of genomic B2-2 DNA, however, were barely detectable above background levels. Therefore, the detection limit with randomly labeled pure genomic DNA under these hybridization conditions was estimated to be approximately 0.2 ng.

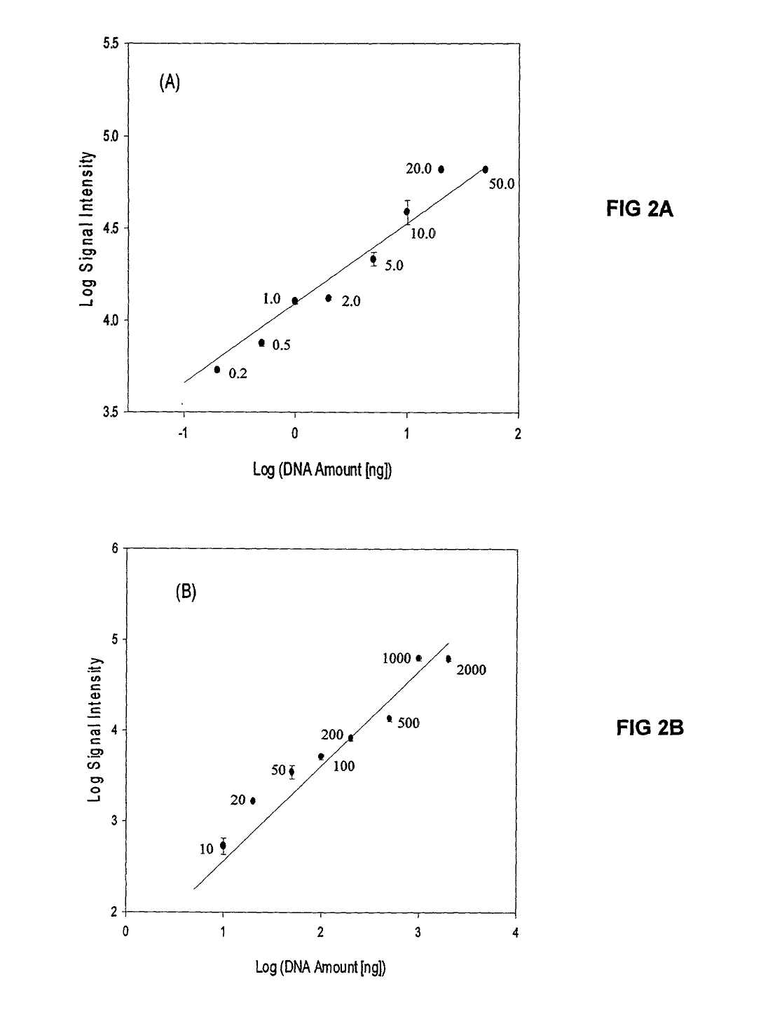

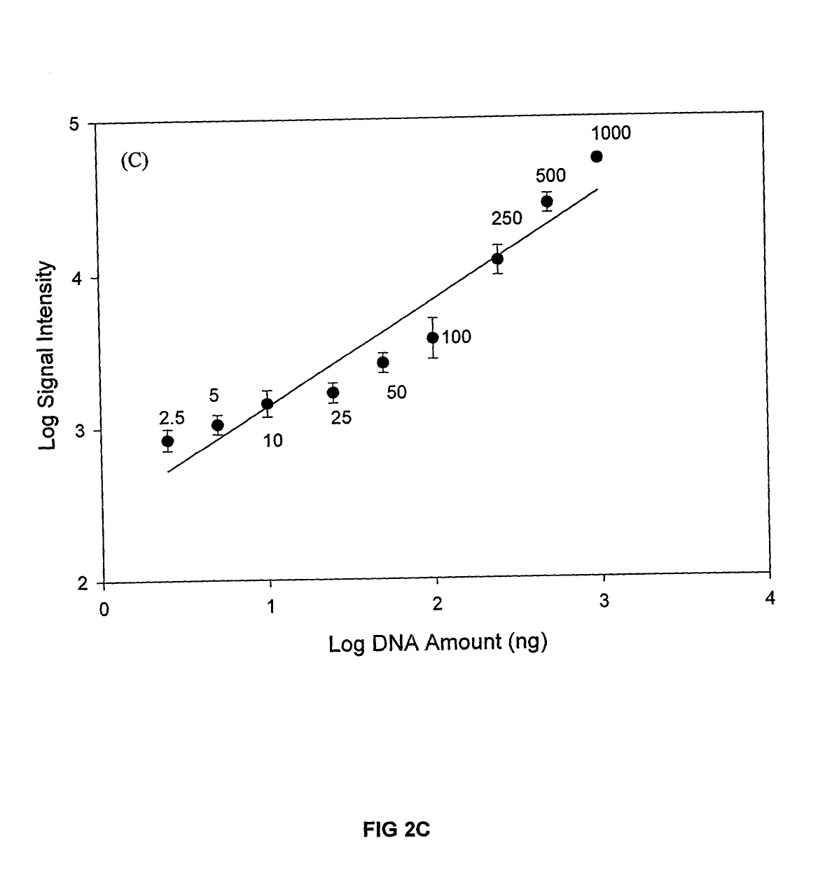

Quantitative potential of CGA hybridization. The assessment of microbial community composition and structure requires the quantification of individual target populations. The capacity of CGA hybridization to serve as a quantitative tool was explored by examining the relationship between the concentration of labeled target DNA and hybridization signal intensity. Genomic DNA from a pure culture of P. stutzeri B2-2 was fluorescently labeled with Cy3 as described and hybridized in triplicate with the community genome array at total concentrations ranging from 0.1 to 2000 ng. Labeled target DNA was hybridized to the community genome array at total concentrations of 0.1 to 2000 ng in a total hybridization solution volume of 3 μl. Because the quantitation of signal intensity is significantly affected by the percentage of laser power and PMT gain, these scanning parameters were adjusted, so that none of the signals in the dynamic range of target DNA concentrations was saturated. The fluorescence intensities obtained at each DNA concentration for 9 data points (3 independent microarrays with 3 replicates on each slide) were averaged, and the log of the concentration was compared to the corresponding log value of the mean fluorescence intensity (FIG. 2: the data points represent mean values derived from 3 independent microarray slides, with 3 replicates on each slides; error bars showing the standard deviation). A strong linear relationship was observed between signal intensity and target DNA concentration in the range of 0.2 to 50 ng (R²=0.95; FIG. 2A) and 10 to 2000 ng (R²=0.97; FIG. 2B).

To determine the quantitative capacity of CGA hybridization in the case of a mixed DNA population, pure genomic DNA from 16 targeted bacteria of different genera and species were mixed at varying DNA concentrations, fluorescently labeled, and hybridized with the community genome array. The sixteen genomic DNAs and their concentrations are as follows: (1) P. stutzeri B2-2, 1000 ng; (2) A. tolulyticus, 500 ng; (3) S. oneidensis MR-1, 250 ng; (4) Halomonas variabilis B9-12, 100 ng; (5) Pseudomonas sp. G179, 50 ng; (6) S. algae Bry, 25 ng; (7) E. coli, 10 ng; (8) an unknown a-proteobacterium C1-4, 5 ng; (9) B. methanolicus F6-2, 2.5 ng; (10) Marinobacter sp. E1-7, 1 ng; (11) S. amazonensis SB2B, 0.5 ng; (12) Staphylococcus saprophyticus D3-16, 0.25 ng; (13) S. woodyi MS32, 0.1 ng; (14) Marinobacter sp. D5-10, 0.05 ng; (15) Shewanella sp. A8-3, 0.025 ng; and (16) Marinobacter sp. C10-5, 0.01 ng. The DNA concentrations and their corresponding fluorescence intensities were converted to log values and plotted as shown in FIG. 2C. A linear relationship (R²=0.92) between signal intensity and DNA concentration was obtained only for concentrations in the range of 2.5 to 1000 ng (FIG. 2C). Although the signals were detectable, CGA hybridization could not differentiate DNA quantities in the population that were less than 2.5 ng.

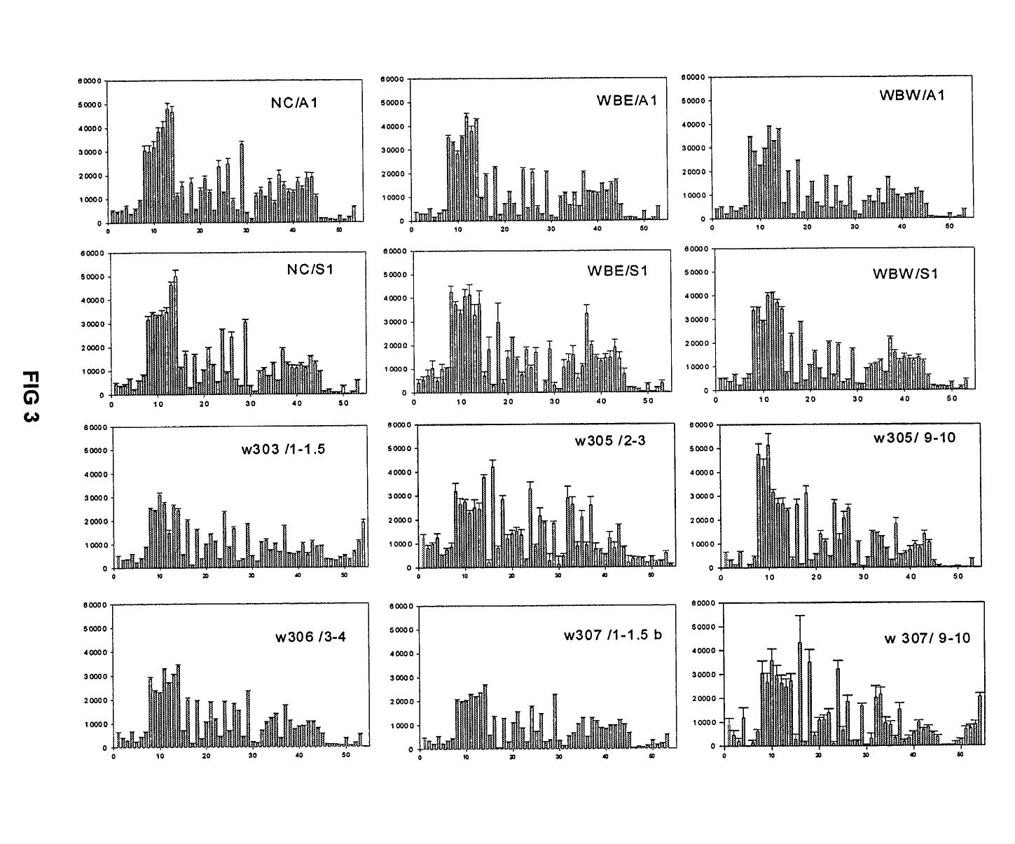

Microarray-based detection of target genomes in environmental samples. To evaluate the potential applicability of DNA microarrays for microbial community analysis, bulk community DNA from soil (NC/A1, WBE/A1, WBW/A1), stream sediment (NC/S1, WBE/S1, WBW/S1), and marine sediment (w303/1-1.5, w305/2-3, w305/9-10, w306/3-4, w307/1-1.5, w307/9-10) samples was directly labeled with Cy5 using the random priming method and hybridized with the CGAs in triplicate. The result is shown in FIG. 3 (the identity of each bar is summarized in Table 3). Strong hybridization signals above background levels were obtained for all of the environmental samples tested. No hybridization with the five yeast control genes was observed. Genome distribution curves indicated that Azoarcus-like species appeared to be dominant in all the environmental samples examined, while P. stutzeri-like organisms were less dominant compared to Azoarcus. Shewanella-like species were detected at very low levels, indicating that their presence was not common in any of the environmental samples tested. The environmental isolate P. stutzeri E4-2 showed variable distribution patterns among the three types of samples.

TABLE 1

| | | | | | | |
|---|---|---|---|---|---|---|
| | | | | DNA | | |
| | | | | Homology | 16S | GyrB |
| | | | | to Labeled | rDNA | Iden- |
| | | Strain/ | | Target | Identity | tity |
| Row | Column | Gene | Source | DNA (%) | (%) | (%) |

Information on Genome Probes and Their Locations on the Microarray

| | | | | | | |
|---|---|---|---|---|---|---|
| 1 | 1 | BrY | Shewanella | 17.6 | 94 | 76.3 |
| | | | algae | | | |
| 2 | 1 | ANG- | S. pealeana | 20.4 | 92.8 | 76.8 |
| | | SQ1 | | | | |
| 3 | 1 | OK-1 | S. algae | 17.6 | 94 | 76.3 |
| 4 | 1 | SB2B | S. | 40.3 | 93 | 77.9 |
| | | | amazonensis | | | |
| 5 | 1 | MS32 | S. woodyi | 39 | 92.8 | 78.6 |
| 6 | 1 | MR-1 | S. oneidensis | 100 | 100 | 100 |
| 7 | 1 | MR-4 | Shewanella | | 97.7 | 90.4 |
| | | | sp. | | | |
| 8 | 1 | Td1 | Azoarcus | 28 | | |
| | | | tolulyticus | | | |
| 9 | 1 | Td2 | A. tolulyticus | 33 | | |
| 10 | 1 | Td3 | A. evansii | 30 | | |
| 11 | 1 | Td15 | A. tolulyticus | | | |
| 12 | 1 | Td17 | A. evansii | 89 | | |
| 13 | 1 | Td19 | A. evansii | 27 | | |
| 14 | 1 | Td21 | A. evansii | 100 | | |
| 15 | 1 | BL-11 | BL-11 | 28 | | |
| 1 | 2 | VB22T | VB22T | | | |
| 2 | 2 | A8-3 | Shewanella | | | |
| | | | sp. | | | |
| 3 | 2 | B2-2 | Pseudomonas | | | |
| | | | stutzeri | | | |
| 4 | 2 | B9-12 | Halomonas | | | |
| | | | variabilis | | | |
| 5 | 2 | C1-4 | α-proteo- | | | |
| | | | bacterium | | | |
| 6 | 2 | C5-1 | P. stutzeri | | | |
| 7 | 2 | C10-5 | Marinobacter | | | |
| | | | sp. | | | |
| 8 | 2 | D3-15 | Marine | | | |
| | | | bacterium | | | |
| 9 | 2 | D3-16 | Staphylococcus | | | |
| | | | saprophyticus | | | |
| 10 | 2 | D5-10 | Marinobacter | | | |
| | | | sp. | | | |
| 11 | 2 | D7-6 | P. stutzeri | | | |
| 12 | 2 | D8-12 | P. stutzeri | | | |
| 13 | 2 | E1-7 | Marinobacter | | | |
| | | | sp. | | | |
| 14 | 2 | E4-2 | P. stutzeri | | | |
| 15 | 2 | F6-2 | Bacillus | | | |
| | | | methanolicus | | | |
| 1 | 3 | F7-3 | Bacillus | | | |
| | | | methanolicus | | | |
| 2 | 3 | F9-1 | P. stutzeri | | | |
| 3 | 3 | F9-2 | P. stutzeri | | | |
| 4 | 3 | 2-25 | Marinobacter | | | |
| | | | sp. | | | |
| 5 | 3 | G179 | Pseudomonas | | | |
| | | | sp. | | | |
| 6 | 3 | ATCC | P. stutzeri | | | |
| | | 17592 | | | | |
| 7 | 3 | DSM | P. stutzeri | | | |
| | | 50238 | | | | |
| 8 | 3 | ATCC | P. stutzeri | | | |
| | | 17594 | | | | |
| 9 | 3 | ATCC | P. stutzeri | | | |
| | | 17595 | | | | |
| 10 | 3 | ATCC | P. stutzeri | | | |
| | | 17587 | | | | |
| 11 | 3 | CCUG | P. stutzeri | | | |
| | | 11256 | | | | |
| 12 | 3 | ATCC | P. stutzeri | | | |
| | | 27591 | | | | |
| 13 | 3 | DNSP21 | P. stutzeri | | | |
| 14 | 3 | DSM | P. balearica | | | |
| | | 6083 | | | | |
| 15 | 3 | S17-1/ | E. coli | | | |
| | | lpir | | | | |
| 1 | 4 | | Saccharomyces | | | |
| | | | cerevisiae | | | |
| 2 | 4 | mfa1 | S. cerevisiae | | | |
| 3 | 4 | mfa2 | S. cerevisiae | | | |
| 4 | 4 | ras1 | S. cerevisiae | | | |
| 5 | 4 | act1 | S. cerevisiae | | | |
| 6 | 4 | ste3 | S. cerevisiae | | | |
| 7 | 4 | 7-1 | Shewanella sp. | | | |
| 8 | 4 | 7-2 | Shewanella sp. | | | |
| 9 | 4 | 11-1 | Shewanella | | | |
| | | | gelidimarina | | | |
| 10 | 4 | 11-2 | Shewanella sp. | | | |
| 11 | 4 | 14-2 | Shewanella sp. | | | |
| 12 | 4 | PS-2 | Shewanella sp. | | | |
| | | (SQ26) | | | | |
| 13 | 4 | PS-3 | Shewanella sp. | | | |
| | | (SQ26) | | | | |
| 14 | 4 | PV-3 | Shewanella | | | |
| | | | woodyi | | | |
| 15 | 4 | blank | blank | | | |

Genome Probes and Their Locations on the Microarray

| Row | Column a | Column b | Column c | Column d |
|---|---|---|---|---|
| 1 | Shewanella | VB22T | B. | Saccharomyces |
| | algae Bry | | methanolicus | cerevisiae |
| | | | F7-3 | |
| 2 | Shewanella | Shewanella sp. | P. stutzeri F9-1 | Yeast mfa1 |
| | pealeana | A8-3 | | gene |
| | ANG-SQ1 | | | |
| 3 | S. algae | Pseudomonas | P. stutzeri F9-2 | Yeast mfa2 |
| | OK-1 | stutzeri B2-2 | | gene |
| 4 | Shewanella | Halomonas | Marinobacter | Yeast ras1 gene |
| | amazonensis | variabilis B9-12 | sp. 2-25 | |
| | SB2B | | | |
| 5 | Shewanella | α- | Pseudomonas | Yeast act1 gene |
| | woodyi | Proteobacterium | sp. G179 | |
| | MS32 | C1-4 | | |
| 6 | Shewanella | P. stutzeri C5-1 | P. stutzeri | Yeast ste3 gene |
| | oneidensis | | ATCC 17592 | |
| | MR-1 | | | |
| 7 | Shewanella | Marinobacter sp. | P. stutzeri | Shewanella sp. |
| | oneidensis | C10-5 | DSM 50238 | 7-1 |
| | MR-4 | | | |
| 8 | Azoarcus | Marine | P. stutzeri | Shewanella sp. |
| | tolulyticus | bacterium D3-15 | ATCC 17594 | 7-2 |
| | Td1 | | | |
| 9 | A. tolulyticus | Staphylococcus | P. stutzeri | Shewanella |
| | Td2 | saprophyticus | ATCC 17595 | gelidimarina |
| | | D3-16 | | 11-1 |
| 10 | Azoarcus | Marinobacter sp. | P. stutzeri | Shewanella sp. |
| | evansii Td3 | D5-10 | ATCC 17587 | 11-2 |
| 11 | A. tolulyticus | P. stutzeri D7-6 | P. stutzeri | Shewanella sp. |
| | Td15 | | CCUG 11256 | 14-2 |
| 12 | A. evansii | P. stutzeri D8-12 | P. stutzeri | Shewanella sp. |
| | Td17 | | ATCC 27591 | PS-2 (SQ26) |
| 13 | A. evansii | Marinobacter sp. | P. stutzeri | Shewanella sp. |
| | Td19 | E1-7 | DNSP21 | P5-3 (SQ26) |
| 14 | A. evansii | P. stutzeri E4-2 | Pseudomonas | Shewanella |
| | Td21 | | balearica DSM | woodyi PV-3 |
| | | | 6083 | |
| 15 | BL-11 | Bacillus | Escherichia | blank |
| | | methanolicus | coli 517-1/lpir | |
| | | F6-2 | | |

TABLE 2
DNA Similarity of Azoarcus strains to Labeled Target Genome Td21^{a}

| | | | | DNA Similarity |
|---|---|---|---|---|
| Row | Column | Strain | Source | to Td21 |
| 8 | a | Td1 | A. tolulyticus | 28 |
| 9 | a | Td2 | A. tolulyticus | 33 |
| 10 | a | Td3 | A. evansii | 30 |
| 11 | a | Td15 | A. tolulyticus | N/A^{b} |
| 12 | a | Td17 | A. evansii | 89 |
| 13 | a | Td19 | A. evansii | 27 |
| 14 | a | Td21 | A. evansii | 100 |
| 15 | a | BL-11 | BL-11 | 28 |

^{a}DNA similarity estimations were based on hybridization experiments and were previously reported by Song et al. (1999).
^{b}N/A, data not available.

TABLE 3
Identity of each bar in FIG. 3.

| | |
|---|---|
| bar1 | Shewanella algae Bry |
| bar2 | S. pealeana ANG-SQ1 |
| bar3 | S. algae OK-1 |
| bar4 | S. amazonensis SB2B |
| bar5 | S. woodyi MS32 |
| bar6 | S. sp. MR-1 |
| bar7 | S. sp. MR-4 |
| bar8 | Azoarcus tolulyticus Td1 |
| bar9 | A. tolulyticus Td2 |
| bar10 | A. evansii Td3 |
| bar11 | A. tolulyticus Td15 |
| bar12 | A. evansii Td17 |
| bar13 | A. evansii Td19 |
| bar14 | A. evansii Td21 |
| bar15 | BL-11 |
| bar16 | VB22T |
| bar17 | Shewanella sp. A8-3 |
| bar18 | Pseudomonas stutzeri B2-2 |
| bar19 | Halomonas vatiabilis B9-12 |
| bar20 | α proteobacterium C1-4 |
| bar21 | P. stutzeri C5-1 |
| bar22 | Marinobacter sp. C10-5 |
| bar23 | Marine Bacterium D3-15 |
| bar24 | Staph. Saprophyticus D3-16 |
| bar25 | Marinobacter sp. D5-10 |
| bar26 | P. stutzeri D7-6 |
| bar27 | P. stutzeri D8-12 |
| bar28 | Marinobacter sp. E1-7 |
| bar29 | P. stutzeri E4-2 |
| bar30 | Bacillus methanolicus F6-2 |
| bar31 | Bacillus methanolicus F7-3 |
| bar32 | P. stutzeri F9-1 |
| bar33 | P. stutzeri F9-2 |
| bar34 | Marinobacter sp. 2-25 |
| bar35 | Pseudomonas sp. G179 |
| bar36 | P. stutzeri 17592 |
| bar37 | P. stutzeri DSM 50238 |
| bar38 | P. stutzeri 17594 |
| bar39 | P. stutzeri 17595 |
| bar40 | P. stutzeri 17587 |
| bar41 | P. stutzeri CCUG 11256 |
| bar42 | P. stutzeri 27591 |
| bar43 | P. stutzeri DNSP21 |
| bar44 | P. balearica DSM 6083 |
| bar45 | E. coli |
| bar46 | Shewanella sp. 7-1 |
| bar47 | Shewanella sp. 7-2 |
| bar48 | Shewanella gelidimarina 11 |
| bar49 | Shewanella sp. 11-2 |
| bar50 | Shewanella sp. 14-2 |
| bar51 | PS-2 (Shewanella sp. SQ26) |
| bar52 | PS-3 (Shewanella sp. SQ26) |
| bar53 | PV-3 (Shewanella sp. Woodyi) |

### REFERENCES

1. Amann, R. I., W. Ludwig, and K.-H. Schleifer. 1995. Phylogenetic identification and in situ detection of individual microbial cells without cultivation. Microbiol. Rev. 59:143-169.

2. Bagwell, C. E., and C. R. Lovell. 2000. Persistence of selected Spartina alterniflora rhizoplane diazotrophs exposed to natural and manipulated environmental variability. Appl. Environ. Microbiol. 66:4625-4633.

3. Braker, G., J. Zhou, L. Wu, A. H. Devol, and J. M. Tiedje. 2000. Nitrite reductase genes (nirK and nirS) as functional markers to investigate diversity of denitrifying bacteria in Pacific Northwest marine sediment communities. Appl. Environ. Microbiol. 66:2096-2104.

4. Brocklehurst, K. R., and A. P. Morby. 2000. Metal-ion tolerance in Escherichia coli: analysis of transcriptional profiles by gene-array technology. Microbiol. 146:2277-2282.

5. Chee, M., R. Yang, E. Hubbell, A. Berno, X. C. Huang, D. Stern, J. Winkler, D. J. Lockhart, M. S. Morris, and S. P. A. Fodor. 1996. Accessing genetic information with high-density DNA arrays. Science 274:610-614.

6. DeRisi, J. L., V. R. Iyer, and P. O. Brown. 1997. Exploring the metabolic and genetic control of gene expression on a genomic scale. Science 278:680-686.

7. DeRisi, J., L. Penland, P. O. Brown, M. L. Bittner, P. S. Meltzer, M. Ray, Y. Chen, Y. A. Su, and J. M. Trent. 1996. Use of a cDNA microarray to analyse gene expression patterns in human cancer. Nat. Genet. 14:457-460.

8. de Saizieu, A., U. Certa, J. Warrington, C. Gray, W. Keck, and J. Mous. 1998. Bacterial transcript imaging by hybridization of total RNA to oligonucleotide arrays. Nat. Biotech. 16:45-48.

9. Drmanac, S., N. A. Stavropulos, I. Labat, J. Vonau, B. Hauser, M. B. Soares, and R. Drmanac. 1996. Gene-representing cDNA clusters defined by hybridization of 57,419 clones from infant brain libraries with short oligonucleotide probes. Genomics 37:29-40.

10. Ferea, T. L., D. Botstein, P. O. Brown, and R. F. Rosenzweig. 1999. Systematic changes in gene expression patterns following adaptive evolution in yeast. Proc. Natl. Acad. Sci. USA 96:9721-9726.

11. Futcher, B. 2000. Microarrays and cell cycle transcription in yeast. Curr. Opin. Cell. Biol. 12:710-715.

12. Gasch, A. P., P. T. Spellman, C. M. Kao, O. Carmel-Harel, M. B. Eisen, G. Storz, D. Botstein, and P. O. Brown. 2000. Genomic expression programs in the response of yeast cells to environmental changes. Mol. Biol. Cell. 11:4241-4257.

13. Gross, C., M. Kelleher, V. R. Iyer, P. O. Brown, and D. R. Winge. 2000. Identification of the copper regulon in Saccharomyces cerevisiae by DNA microarrays. J. Biol. Chem. 275:32310-32316.

14. Hacia, J. G. 1999. Resequencing and mutational analysis using oligonucleotide microarrays. Nat. Genet. 21:42-47.

15. Heller, R. A., M. Schena, A. Chai, D. Shalon, T. Bedilion, J. Gilmore, D. E. Woolley, and R. W. Davis. 1997. Discovery and analysis of inflammatory disease-related genes using cDNA microarrays. Proc. Natl. Acad. Sci. USA 94:2150-2155.

16. Kalogeraki, V. S., and S. C. Winans. 1997. Suicide plasmids containing promoterless reporter genes can simultaneously disrupt and create fusions to target genes of diverse bacteria. Gene 188:69-75.

17. Khodursky, A. B., B. J. Peter, N. R. Cozzarelli, D. Botstein, P. O. Brown, and C. Yanofsky. 2000. DNA microarray analysis of gene expression in response to physiological and genetic changes that affect tryptophan metabolism in Escherichia coli. Proc. Natl. Acad. Sci. USA 97:12170-12175.

18. Lashkari, D. A., J. L. DeRisi, J. H. McCusker, A. F. Namath, C. Genetile, S. Y. Hwang, P. O. Brown, and R. W. Davis. 1997. Yeast microarrays for genome wide parallel genetic and gene expression analysis. Proc. Natl. Acad. Sci. USA 94:13057-13062.

19. Lockhart, D. J., H. Dong, M. C. Byrne, M. T. Follettie, M. V. Gallo, M. S. Chee, M. Mittmann, C. Wang, M. Kobayashi, H. Horton, and E. L. Brown. 1996. Expression monitoring by hybridization to high-density oligonucleotide arrays. Nat. Biotech. 14:1675-1680.

20. Lyons, T. J., A. P. Gasch, L. A. Gaither, D. Botstein, P. O. Brown, and D. J. Eide. 2000. Genome-wide characterization of the Zap1p zinc-responsive regulon in yeast. Proc. Natl. Acad. Sci. USA 97:7957-7962.

21. Milosavljevic, A., S. Savkovic, R. Crkvenjakov, D. Salbego, H. Serrato, H. Kreuzer, A. Gemmell, S. Batus, D. Grujic, S. Carnahan, T. Paunesku, and J. Tepavcevic. 1996. DNA sequence recognition by hybridization to short oligomers: experimental verification of the method on the E. coli genome. Genomics 37:77-86.

22. Peterson, S., R. T. Cline, H. Tettelin, V. Sharov, and D. A. Morrison. 2000. Gene expression analysis of the Streptococcus pneumoniae competence regulons by use of DNA microarrays. J. Bacteriol. 182:6192-6202.

23. Ramsay, G. 1998. DNA chips: state-of-the art. Nat. Biotech. 16:40-44.

24. Richmond, C. S., J. D. Glasner, R. Mau, H. Jin, and F. R. Blattner. 1999. Genome-wide expression profiling in Escherichia coli K-12. Nucl. Acids Res. 27:3821-3835.

25. Rossello, R., E. Garcia-Valdes, J. Lalucat, and J. Ursing. 1991. Genotypic and phenotypic diversity of Pseudomonas stutzeri. System. Appl. Microbiol. 14:150-157.

26. Sambrook, J., E. F. Fritsch, and T. Maniatis. 1989. Molecular cloning: a laboratory manual, 2nd ed. Cold Spring Harbor Laboratory Press, Cold Spring Harbor, N.Y.

27. Schena, M., D. Shalon, R. W. Davis, and P. O. Brown. 1995. Quantitative monitoring of gene expression patterns with a complementary DNA microarray. Science 270:467-470.

28. Schena, M., D. Shalon, R. Heller, A. Chai, P. O. Brown, and R. W. Davis. 1996. Parallel human genome analysis: microarray-based expression monitoring of 1000 genes. Proc. Natl. Acad. Sci. USA 93:10614-10619.

29. Selinger D. W., K. J. Cheung, R. Mei, E. M. Johansson, C. S. Richmond, F. R. Blattner, D. J. Lockhart, and G. M. Church. 2000. RNA expression analysis using a 30 base pair resolution Escherichia coli genome array. Nat. Biotech. 18:1262-1268.

30. Shalon, D., S. J. Smith, and P. O. Brown. 1996. A DNA microarray system for analyzing complex DNA samples using two-color fluorescent probe hybridization. Genome Res. 6:639-645.

31. Shen, Y., L. G. Stehmeier, and G. Voordouw. 1998. Identification of hydrocarbon-degrading bacteria in soil by reverse sample genome probing. Appl. Environ. Microbiol. 64:637-645.

32. Song, B., M. M. Haggblom, J. Z. Zhou, J. M. Tiedje, and N. J. Palleroni. 1999. Taxonomic characterization of denitrifying bacteria that degrade aromatic compounds and description of Azoarcus toluvorans sp nov and Azoarcus toluclasticus sp nov. Int. J. Syst. Bacteriol. 49:1129-1140.

33. Sudarsanam, P., V. R. Iyer, P. O. Brown, and F. Winston. 2000. Whole-genome expression analysis of snf/swi mutants of Saccharomyces cerevisiae. Proc. Natl. Acad. Sci. USA 97:3364-3369.

34. Venkateswaran, K., D. P. Moser, M. E. Dollhopf, D. P. Lies, D. A. Saffarini, B. J. MacGregor, D. B. Ringelberg, D. C. White, M. Nishijima, H. Sano, J. Burghardt, E. Stackebrandt, and K. H. Nealson. 1999. Polyphasic taxonomy of the genus Shewanella and description of Shewanella oneidensis sp. nov. Int. J. Syst. Bacteriol. 49:705-724.

35. Voordouw, G., Y. Shen, C. S. Harrington, A. J. Telang, T. R. Jack, and D. W. S. Westlake. 1993. Quantitative reverse sample genome probing of microbial communities and its application to oil field production waters. Appl. Environ. Microbiol. 59:4101-4114.

36. Voordouw, G., J. K. Voordouw, T. R. Jack, J. Foght, P. M. Fedorak, and D. W. S. Westlake. 1992. Identification of distinct communities of sulfate-reducing bacteria in oil fields by reverse sample genome probing. Appl. Environ. Microbiol. 58:3542-3552.

37. Voordouw, G., J. K. Voordouw, R. R. Karkhoff-Schweizer, P. M. Fedorak, and D. W. S. Westlake. 1991. Reverse sample genome probing, a new technique for identification of bacteria in environmental samples by DNA hybridization, and its application to the identification of sulfate-reducing bacteria in oil field samples. Appl. Environ. Microbiol. 57:3070-3078.

38. Wang, D. G., J. B. Fan, C. J. Siao, A. Berno, P. Young, R. Sapolsky, G. Ghandour, N. Perkins, E. Winchester, J. Spencer, L. Kruglyak, L. Stein, L. Hsie, T. Topaloglou, E. Hubbell, E. Robinson, M. Mittmann, M. S. Morris, N. Shen, D. Kilburn, J. Rioux, C. Nusbaum, S. Rozen, T. J. Hudson, R. Lipshutz, M. Chee, and E. S. Lander. 1998. Large-scale identification, mapping, and genotyping of single-nucleotide polymorphisms in the human genome. Science 280:1077-1082.

39. Wei Y., J. M. Lee, C. Richmond, F. R. Blattner, J. A. Rafalski, and R. A. LaRossa. 2001. High-density microarray-mediated gene expression profiling of Escherichia coli. J. Bacteriol. 183:545-556.

40. White, K. P., S. A. Rifkin, P. Hurban, and D. S. Hogness. 1999. Microarray analysis of Drosophila development during metamorphosis. Science 286:2179-2184.

41. Wodicka, L., H. Dong, M. Mittmann, M.-H. Ho, and D. J. Lockhart. 1997. Genome-wide expression monitoring in Saccharomyces cerevisiae. Nature Biotech. 15:1359-1367.

42. Wu, L., D. K. Thompson, G. Li, R. A. Hurt, J. M. Tiedje, and J. Zhou. 2001. Development and evaluation of functional gene arrays for detection of selected genes in the environment. Appl. Environ. Microbiol. 67:5780-5790.

43. Ye, R. W., W. Tao, L. Bedzyk, T. Young, M. Chen, and L. Li. 2000. Global gene expression profiles of Bacillus subtilis grown under anaerobic conditions. J. Bacteriol. 182:4458-4465.

44. Zhang, L., W. Zhou, V. E. Velculescu, S. E. Kern, R. H. Hruban, S. R. Hamilton, B. Vogelstein, and K. W. Kinzler. 1997. Gene expression profiles in normal and cancer cells. Science 276:1268-1272.

45. Zhou, J.-Z., M. A. Bruns, and J. M. Tiedje. 1996. DNA recovery from soils of diverse composition. Appl. Environ. Microbiol. 62:316-322.

We claim:

1. A method for determining the presence or absence of a microorganism in a sample comprising the steps of:
providing a nucleic acid microarray wherein the microarray comprises one or more probes for the microorganism wherein the one or more probes comprise at least 90% of the whole genomic DNA or RNA of the microorganism and are located at one particular position on the microarray;
providing a labeled DNA or RNA preparation derived from the sample;
hybridizing the labeled DNA or RNA preparation to the microarray;
washing the microarray; and
observing the presence or absence of a hybridization signal at the particular position where the one or more probes are located to determine the presence or absence of the microorganism in the sample.

2. The method of claim 1, wherein the one or more probes comprise at least 95% of the whole genomic DNA or RNA of the microorganism.

3. The method of claim 1, wherein the one or more probes comprise at least 97% of the whole genomic DNA or RNA of the microorganism.

4. The method of claim 1, wherein the one or more probes comprise at least 99% of the whole genomic DNA or RNA of the microorganism.

5. The method of claim 1, wherein the one or more probes comprise 100% of the whole genomic DNA or RNA of the microorganism.

6. The method of claim 1, wherein the number of the probes for a microorganism on the microarray is identical to the number of chromosomes the microorganism has.

7. The method of claim 6, wherein the number of the probes for a microorganism on the microarray is one.

8. The method of claim 1, wherein the microarray is a DNA microarray.

9. The method of claim 1, wherein the microarray is a RNA microarray.

10. The method of claim 1, wherein the microorganism is selected from viruses, bacteria, yeasts, fungi and algae.

11. The method of claim 1, wherein the microorganism is a virus.

12. The method of claim 1, wherein the microorganism is a bacterium.

13. The method of claim 1, wherein the microorganism is a yeast.

14. The method of claim 1, wherein the microorganism is a fungus.

15. The method of claim 1, wherein the hybridization is conducted in a buffer containing about 5% to about 70% formamide.

16. The method of claim 1, wherein the hybridization is conducted in a buffer containing about 30% to about 70% formamide.

17. The method of claim 1, wherein the hybridization is conducted in a buffer containing about 50% to about 70% formamide.

18. The method of claim 1, wherein the hybridization is conducted at a temperature from about 45° C. to about 75° C.

19. The method of claim 1, wherein the hybridization is conducted at a temperature from about 55° C. to about 75° C.

20. The method of claim 1, wherein the post-hybridization washing is conducted with a buffer containing 0×SSC to about 0.1×SSC.

21. The method of claim 1, wherein the post-hybridization washing is conducted with a buffer containing 0×SSC to about 0.05×SSC.

22. The method of claim 1, wherein the post-hybridization washing is conducted with a buffer containing 0×SSC to about 0.01×SSC.

23. The method of claim 1, wherein the labeled DNA or RNA preparation derived from the sample is fluorescently labeled.

24. The method of claim 23, wherein the labeled DNA or RNA preparation derived from the sample is labeled by a compound selected from Cy3, Cy5, Cy3.5, Cy5.5, and Alexa fluorescence dyes.

25. The method of claim 24, wherein the labeled DNA or RNA preparation derived from the sample is labeled by a compound selected from Cy3 and Cy5.

26. A polynucleotide microarray comprising one or more probes for a microorganism wherein the one or more probes comprise at least 90% of the whole genomic DNA or RNA of the microorganism and are located at one particular position of the microarray.

27. The microarray of claim 26, wherein the one or more probes comprise at least 95% of the whole genomic DNA or RNA of the microorganism.

28. The microarray of claim 26, wherein the one or more probes comprise at least 97% of the whole genomic DNA or RNA of the microorganism.

29. The microarray of claim 26, wherein the one or more probes comprise at least 99% of the whole genomic DNA or RNA of the microorganism.

30. The microarray of claim 26, wherein the one or more probes comprise 100% of the whole genomic DNA or RNA of the microorganism.

31. The method of claim 26, wherein the number of the probes for a microorganism on the microarray is identical to the number of chromosomes the microorganism has.

32. The method of claim 31, wherein the number of the probes for a microorganism on the microarray is one.

33. The microarray of claim 26, wherein the microarray is a DNA microarray.

34. The microarray of claim 26, wherein the microarray is a RNA microarray.

35. The microarray of claim 26, wherein the microorganism is selected from viruses, bacteria, yeasts, fungi and algae.

36. The microarray of claim 26, wherein the microorganism is a virus.

37. The microarray of claim 26, wherein the microorganism is a bacterium.

38. The microarray of claim 26, wherein the microorganism is a yeast.

39. The microarray of claim 26, wherein the microorganism is a fungus.

40. A method for building a nucleic acid microarray comprising the steps of isolating the whole genomic DNA or RNA from a microorganism and spotting the whole genomic DNA or RNA onto a particular position of microarray substrate.
